(19) United States
(12) Patent Application Publication
(10) Pub. No.: US 2010/0312273 A1
(43) Pub. Date: Dec. 9, 2010

(54) COMBINED VASCULAR AND CLOSURE SHEATH

(76) Inventor: Robert E. Kim, Acworth, GA (US)

Correspondence Address: CROSE LAW LLC, 112 NORTHBROOKE TRACE, WOODSTOCK, GA, 30188, US

(21) Appl. No.: 12/477,203

(22) Filed: Jun. 3, 2009

(51) Int. Cl. A61B 17/03

(52) U.S. Cl. 606/214; 606/213

(57) ABSTRACT

The technology described herein provides an apparatus and associated methods for the rapid percutaneous occlusion of a vascular access site. In one exemplary embodiment, a combined vascular and closure sheath is provided. This device includes a vascular sheath having a vascular sheath lumen, a proximal end and a distal end, and an exterior surface. The combined vascular and closure sheath includes a closure sheath, being lesser in length and greater in diameter than the vascular sheath and having a proximal end and a distal end, disposed circumferentially around the exterior surface of the vascular sheath at the proximal end of the vascular sheath, thereby forming a closure sheath lumen between the exterior surface of the vascular sheath and an interior surface of the closure sheath, the closure sheath being configured operably to eject a fluid such as a coagulant, a tissue sealant, and the coagulation protein thrombin.

### FIELD OF THE INVENTION

The technology described herein relates generally to the fields of medical devices and percutaneous vascular procedures. More specifically, this technology relates to an apparatus and associated methods for the rapid percutaneous occlusion of a vascular access site.

### BACKGROUND OF THE INVENTION

Surgical procedures, such as catheterization, for example, exist in which medical specialists utilize percutaneous entry into a blood vessel or body cavity. Vascular access generally requires a puncture to a vessel wall. By way of example, a hollow needle can be utilized to pass through skin, muscle tissue, and the like, to access the vascular system. Upon accessing the vascular system, a guide wire and a vascular sheath can be utilized to be placed within the vessel for subsequent catheterization. Thus, the vascular sheath is useful for the insertion of various devices into the blood vessel for therapeutic, diagnostic, and interventional procedures.

At the conclusion of the surgical procedure, when the various devices and the vascular sheath are removed, the puncture site remains in the blood vessel. To avoid complications, such as hematoma, subsequent steps must be taken to ensure the proper and timely closure of the puncture site. Prolonged manual pressure is often utilized when natural blood clotting can be relied upon. Additionally, the utilization of collagen plugs, biodegradable clips, and sutures are known techniques to close the puncture site.

Related patents known in the art include the following: U.S. Pat. No. 6,045,570, issued to Epstein et al. on Apr. 4, 2000, discloses a biological sealant mixture and system for use in percutaneous occlusion of puncture sites and tracts in the human body. U.S. Pat. No. 6,296,658, issued to Gershony et al. on Oct. 2, 2001, discloses a vascular sealing apparatus. U.S. Pat. No. 6,461,364, issued to Ginn et al. on Oct. 8, 2002, discloses a vascular sheath with bioabsorbable puncture site closure apparatus and methods of use.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

In various exemplary embodiments, the technology described herein provides an apparatus and associated methods for the rapid percutaneous occlusion of a vascular access site.

In one exemplary embodiment, the technology described herein provides a combined vascular and closure sheath. The combined vascular and closure sheath includes a vascular sheath having a vascular sheath lumen, a proximal end and a distal end, and an exterior surface, the vascular sheath being generally cylindrical. The combined vascular and closure sheath includes a closure sheath, being lesser in length and greater in diameter than the vascular sheath and having a proximal end and a distal end, disposed circumferentially around the exterior surface of the vascular sheath at the proximal end of the vascular sheath, thereby forming a closure sheath lumen between the exterior surface of the vascular sheath and an interior surface of the closure sheath, the closure sheath being configured operably to eject a fluid.

In at least one embodiment, the fluid to be ejected from the closure sheath can be a coagulant, a tissue sealant, the coagulation protein thrombin, or the like.

The combined vascular and closure sheath can also include a fluid injection port disposed upon the closure sheath and through which the fluid is injected into the closure sheath lumen.

The combined vascular and closure sheath can also include a plurality of apertures disposed upon the distal end of the closure sheath and through which the fluid operably is ejected. As such, the distal end and the proximal end of the closure sheath are sealably connected to the exterior surface of the vascular sheath to enclose the closure sheath lumen and to direct a flow of the fluid through the closure sheath lumen in order to exit the plurality of apertures.

The combined vascular and closure sheath can also include at least one radiopaque marker to provide for a correct alignment of the closure sheath relative to a vessel wall prior to ejection of the fluid through the closure sheath. In at least one embodiment, the at least one radiopaque marker includes a first radiopaque marker disposed at the distal end of the vascular sheath and a second radiopaque marker disposed on the vascular sheath immediately below the distal end of the closure sheath.

The combined vascular and closure sheath can also include at least one ultrasound marker to provide for a correct alignment of the closure sheath relative to a vessel wall prior to ejection of the fluid through the closure sheath. In at least one embodiment, the at least one ultrasound marker includes a first ultrasound marker disposed at the distal end of the vascular sheath and a second ultrasound marker disposed on the vascular sheath immediately below the distal end of the closure sheath.

In another exemplary embodiment, the technology described herein provides a method for sealing a puncture in a vessel wall. The method includes utilizing a combined vascular and closure sheath comprising a vascular sheath having a vascular sheath lumen, a proximal end and a distal end, and an exterior surface, the vascular sheath being generally cylindrical, and comprising a closure sheath, being lesser in length and greater in diameter than the vascular sheath and having a proximal end and a distal end, disposed circumferentially around the exterior surface of the vascular sheath at the proximal end of the vascular sheath, thereby forming a closure sheath lumen between the exterior surface of the vascular sheath and an interior surface of the closure sheath, the closure sheath being configured operably to eject a fluid. The method includes inserting the distal end of the combined vascular and closure sheath through the skin of a patient and into a vessel through a puncture in the vessel. The method includes performing a diagnostic or therapeutic procedure within the vessel by advancing an apparatus through the vascular sheath lumen. The method includes withdrawing the apparatus in the vascular sheath lumen. The method includes ejecting the fluid from the closure sheath to the puncture in the vessel wall, and not within the vessel wall and vascular lumen. The method includes withdrawing the combined vascular and closure sheath.

In at least one embodiment of this method, the combined vascular and closure sheath further includes a fluid injection port disposed upon the closure sheath and through which a fluid is injected into the closure sheath lumen, and the method further includes injecting the fluid through the fluid injection port into the closure sheath lumen.

In at least one embodiment of this method, the combined vascular and closure sheath further includes a plurality of apertures disposed upon the distal end of the closure sheath and through which the fluid operably is ejected, and the method further includes ejecting the fluid through the plurality of apertures to the puncture in the vessel wall, and not within the vessel wall and vascular lumen.

In at least one embodiment of this method, the combined vascular and closure sheath further includes at least one radiopaque marker to provide for a correct alignment of the closure sheath relative to the vessel wall prior to ejection of the fluid through the closure sheath, and the method further includes utilizing a fluoroscope to recognize the at least one radiopaque marker and confirming alignment of the closure sheath relative to the vessel wall prior to ejection of the fluid through the closure sheath. In at least one embodiment of this method, the at least one radiopaque marker includes a first radiopaque marker disposed at the distal end of the vascular sheath and a second radiopaque marker disposed on the vascular sheath immediately below the distal end of the closure sheath.

In at least one embodiment of this method, the combined vascular and closure sheath further includes at least one ultrasound marker to provide for a correct alignment of the closure sheath relative to a vessel wall prior to ejection of the fluid through the closure sheath, and the method further includes utilizing an ultrasound device to recognize the at least one ultrasound marker and confirming alignment of the closure sheath relative to the vessel wall prior to ejection of the fluid through the closure sheath. In at least one embodiment, the at least one ultrasound marker includes a first ultrasound marker disposed at the distal end of the vascular sheath and a second ultrasound marker disposed on the vascular sheath immediately below the distal end of the closure sheath.

In at least one embodiment, the fluid to be ejected from the closure sheath can be a coagulant, a tissue sealant, the coagulation protein thrombin, or the like.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a perspective view of a combined vascular and closure sheath, according to an embodiment of the technology described herein, illustrating, in particular, placement of the device at a vascular access site in a human body;

FIG. 2 is a view of the combined vascular and closure sheath, illustrating, in particular, the vascular port and the thrombin injection port;

FIG. 3 is a view of the combined vascular and closure sheath, illustrating, in particular, placement of the device in a vessel of the vascular system;

FIG. 4A is a view of the combined vascular and closure sheath, inserted through skin surface, muscle tissue, and vessel wall for a diagnostic or therapeutic procedure;

FIG. 4B is a view of the combined vascular and closure sheath depicted in FIG. 4A, illustrating, in particular, the injection of thrombin through the thrombin sheath, once the diagnostic or therapeutic procedure is completed, in order to occlude the vascular access site;

FIG. 4C is a view of the combined vascular and closure sheath depicted in FIG. 4A, illustrating, in particular, the removal of the device through the vessel wall puncture site, tissue, and skin surface opening; and

FIG. 5 is a cross-sectional view of the combined vascular and closure sheath, illustrating, in particular, the vascular sheath lumen and the thrombin sheath lumen.

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides an apparatus and associated methods for the rapid percutaneous occlusion of a vascular access site.

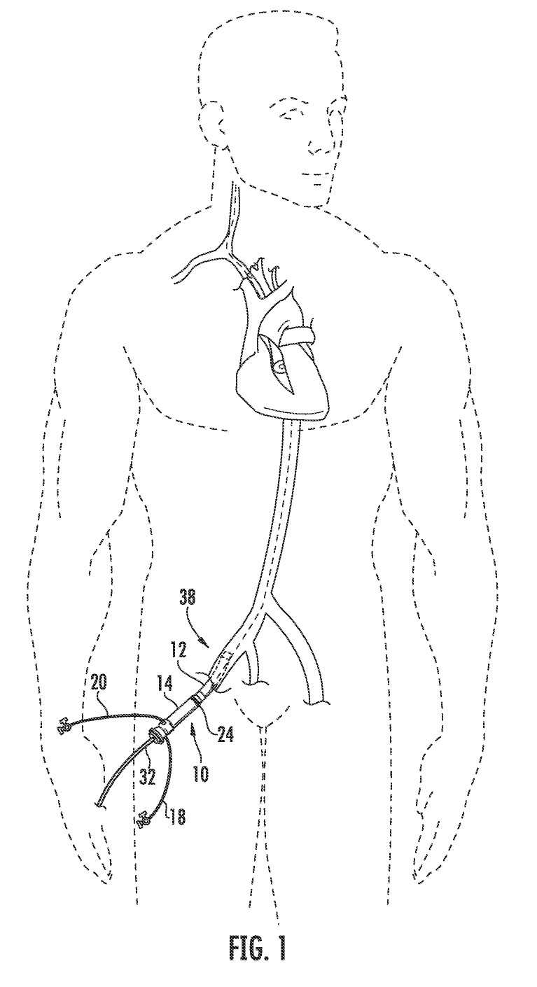

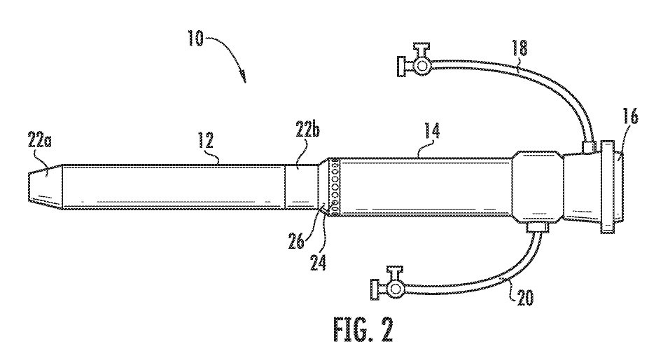

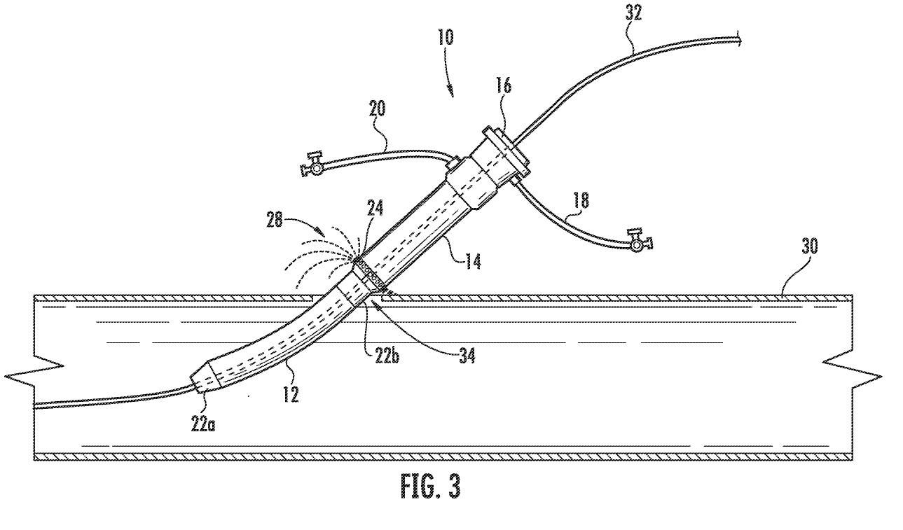

Referring now to FIGS. 1 through 5, a combined vascular and closure sheath 10 is shown. The combined vascular and closure sheath 10 includes a vascular sheath 12 having a vascular sheath lumen 40, a proximal end and a distal end, an exterior surface, vascular port 18, and a hemostatic valve 16. The vascular sheath 12 is generally cylindrical. The vascular sheath 12 is useful in surgical procedures requiring vascular access, such as catheterization and stenting, and the insertion of various devices into the blood vessel for therapeutic, diagnostic, and interventional procedures. Upon accessing the vascular system through a vascular access site, such as area 38 depicted in FIG. 1, a guide wire 32 and a vascular sheath 12 can be utilized to be placed within the vessel for subsequent catheterization or other procedure. Area 38 depicts a common area utilized by medical specialists for vascular access. However, as will be apparent to those of ordinary skill in the art, the combined vascular and closure sheath 10 can be utilized in other locations and other body cavities.

The combined vascular and closure sheath 10 includes a closure sheath 14. The closure sheath 14 is lesser in length and greater in diameter than the vascular sheath 12. The closure sheath 14 has a proximal end and a distal end. The closure sheath 14 is disposed circumferentially around the exterior surface of the vascular sheath 12 at the proximal end of the vascular sheath 12, thereby forming a closure sheath lumen 42 between the exterior surface of the vascular sheath 12 and an interior surface of the closure sheath 14. The closure sheath 14 is thereby configured operably to eject a fluid 28 to the vessel puncture site 34.

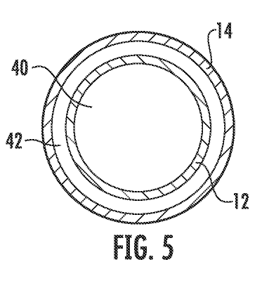

As depicted specifically in FIG. 5, a cross-sectional view of the combined vascular and closure sheath 10 is shown. Within the vascular sheath 12 the vascular sheath lumen 40 provides a pathway for vascular access within a vessel. Within the closure sheath 14, that circumferentially surrounds vascular sheath 12, the closure sheath lumen 42 provides a pathway for the fluid 28 to pass to the puncture site 34 at the appropriate time and as initiated by the medical specialist.

The combined vascular and closure sheath 10 includes a fluid injection port 20 disposed upon the closure sheath 14. Through the fluid injection port 20 the fluid 28 is injected into the lumen of the closure sheath 14. The fluid 28 is prevented from entering the vascular sheath 12.

In at least one embodiment, the fluid to be ejected from the closure sheath can be a coagulant, a tissue sealant, the coagulation protein thrombin, or the like. For the purposes of this disclosure, the coagulation protein thrombin will be utilized; however, as will be apparent to those of ordinary skill in the art, the combined vascular and closure sheath 10 is configured to contain one or more of many fluids known in the art and utilized for one or more of many medical applications. As such, the combined vascular and closure sheath 10 is advantageous in that in reduces the overall amount of medical devices required to provide a therapeutic, diagnostic, and/or interventional procedure through a vascular access site 38 and closure procedures for the vessel puncture 34 in the vascular access site 38. The coagulation protein thrombin provides for the rapid coagulation of the vessel puncture 34. The coagulation protein thrombin is ejected to the vessel puncture 34 by the combined vascular and closure sheath 10 already present at the vessel puncture 34 for use in the medical procedure.

The combined vascular and closure sheath 10 includes a plurality of apertures 24 disposed upon the distal end of the closure sheath 14, immediately above taper 26, and through which the fluid 28, such as the coagulation protein thrombin, operably is ejected. The distal end and the proximal end of the closure sheath 14 are sealably connected to the exterior surface of the vascular sheath 12 to enclose the lumen 42 of the closure sheath 14 and to direct a flow of the fluid 28 through the lumen 42 of the closure sheath 14 in order to exit the plurality of apertures 24.

The combined vascular and closure sheath 10 can also include radiopaque and/or ultrasound markers 22a, 22b, interchangeably. By way of example, the combined vascular and closure sheath 10 can include a radiopaque marker to provide for a correct alignment of the closure sheath 14 relative to a vessel wall 30 prior to ejection of the fluid 28 through the closure sheath 14. In at least one embodiment, a first radiopaque marker 22a is disposed at the distal end of the vascular sheath 12 and a second radiopaque marker 22b is disposed on the vascular sheath 12 immediately below the distal end of the closure sheath 14.

Also by way of example, the combined vascular and closure sheath 10 can include an ultrasound marker to provide for a correct alignment of the closure sheath 14 relative to a vessel wall 30 prior to ejection of the fluid 28 through the closure sheath 14. In at least one embodiment, a first ultrasound marker 22a disposed at the distal end of the vascular sheath 12 and a second ultrasound marker 22b disposed on the vascular sheath 12 immediately below the distal end of the closure sheath 14.

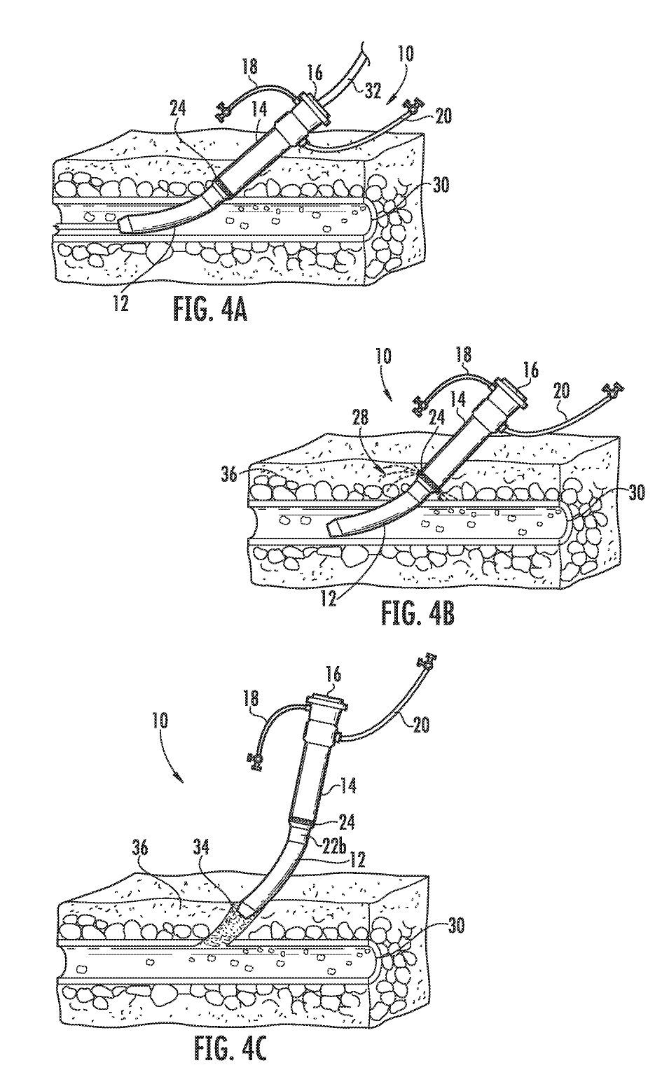

As depicted in FIGS. 4A, 4B, and 4C, use of the combined vascular and closure sheath 10 is illustrated. A hollow needle (not shown) can be utilized to pass through skin, muscle tissue 36, and the like, to access the vascular system by penetrating the vessel wall 30. Upon accessing the vascular system, a guide wire 32 and a vascular sheath 12 can be utilized to be placed within the vessel wall 30 for subsequent catheterization, or other procedure. In FIG. 4A, the combined vascular and closure sheath 10 is shown in use at the vascular access site 38 at vessel puncture 34. The vascular sheath 12 is useful for the insertion of various devices into the blood vessel wall 30 for therapeutic, diagnostic, and interventional procedures.

At the conclusion of the surgical procedure, and while the combined vascular and closure sheath 10 remains at the puncture site 34, markers 22a, 22b can be utilized under a fluoroscope and/or ultrasound, dependent upon the type of markers, and with injection of contrast, to confirm the position of the closure sheath 14. As depicted in FIG. 4B, proper alignment of the closure sheath 14, and its plurality of apertures 24, is critical in order to make certain that any thrombin 28 ejected is outside of the vessel wall 30 and not within the vessel lumen. The guide wire 32, or other medical devices utilized in the vascular lumen for the procedure can be withdrawn, save the combined vascular and closure sheath 10 which remains for the closure process.

The upper marker 22b is positioned just outside of the vascular wall 30. The fluid injection port 20, containing the thrombin 28, is aspirated. This placement assures the plurality of apertures 24 through which the thrombin is ejected are not within the vascular lumen.

As depicted in FIG. 4C, once the thrombin 28 has been ejected from the closure sheath 14, the combined vascular and closure sheath 10 can be removed. The thrombin provides rapid coagulation to the vessel wall 30 puncture site 34. Pressure is to be applied.

In operation, and by way of example, the combined vascular and closure sheath 10, as disclosed and claimed, can be utilized by the following methods steps:
utilizing a combined vascular and closure sheath;
inserting the distal end of the combined vascular and closure sheath through the skin of a patient and into a vessel through a puncture in the vessel;
performing a diagnostic or therapeutic procedure within the vessel by advancing an apparatus through the vascular sheath lumen;
withdrawing the apparatus in the vascular sheath lumen;
ejecting thrombin from the closure sheath to the puncture in the vessel wall, and not within the vessel wall and vascular lumen; and
withdrawing the combined vascular and closure sheath.

Additional method steps can include:
injecting the fluid through the fluid injection port into the closure sheath lumen;
ejecting the fluid through the plurality of apertures to the puncture in the vessel wall, and not within the vessel wall and vascular lumen;
utilizing a fluoroscope to recognize a radiopaque marker;
utilizing an ultrasound device to recognize a radiopaque marker;
confirming alignment of the closure sheath relative to the vessel wall prior to ejection of the fluid through the closure sheath;
utilizing a tissue sealant;
utilizing a coagulant; and
utilizing the coagulation protein thrombin.

It will be apparent to those skilled in the art, after reading this disclosure, which methods steps disclosed can be performed simultaneously or in a different order than that depicted, or omitted given the nature of a particular procedure.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. A combined vascular and closure sheath comprising:
a vascular sheath having a vascular sheath lumen, a proximal end and a distal end, and an exterior surface, the vascular sheath being generally cylindrical; and
a closure sheath, being lesser in length and greater in diameter than the vascular sheath and having a proximal end and a distal end, disposed circumferentially around the exterior surface of the vascular sheath at the proximal end of the vascular sheath, thereby forming a closure sheath lumen between the exterior surface of the vascular sheath and an interior surface of the closure sheath, the closure sheath being configured operably to eject a fluid.

2. The combined vascular and closure sheath of claim 1, further comprising:
a fluid injection port disposed upon the closure sheath and through which the fluid is injected into the closure sheath lumen.

3. The combined vascular and closure sheath of claim 1, further comprising:
a plurality of apertures disposed upon the distal end of the closure sheath and through which the fluid operably is ejected.

4. The combined vascular and closure sheath of claim 4, further comprising:
wherein the distal end and the proximal end of the closure sheath are sealably connected to the exterior surface of the vascular sheath to enclose the closure sheath lumen and to direct a flow of the fluid through the closure sheath lumen in order to exit the plurality of apertures.

5. The combined vascular and closure sheath of claim 1, wherein the fluid to be ejected from the closure sheath is a coagulant.

6. The combined vascular and closure sheath of claim 1, wherein the fluid to be ejected from the closure sheath is a tissue sealant.

7. The combined vascular and closure sheath of claim 1, wherein the fluid to be ejected from the closure sheath is coagulation protein thrombin.

8. The combined vascular and closure sheath of claim 1, further comprising:
at least one radiopaque marker to provide for a correct alignment of the closure sheath relative to a vessel wall prior to ejection of the fluid through the closure sheath.

9. The combined vascular and closure sheath of claim 8, wherein the at least one radiopaque marker comprises a first radiopaque marker disposed at the distal end of the vascular sheath and a second radiopaque marker disposed on the vascular sheath immediately below the distal end of the closure sheath.

10. The combined vascular and closure sheath of claim 1, further comprising:
at least one ultrasound marker to provide for a correct alignment of the closure sheath relative to a vessel wall prior to ejection of the fluid through the closure sheath.

11. The combined vascular and closure sheath of claim 10, wherein the at least one ultrasound marker comprises a first ultrasound marker disposed at the distal end of the vascular sheath and a second ultrasound marker disposed on the vascular sheath immediately below the distal end of the closure sheath.

12. A method for sealing a puncture in a vessel wall, the method comprising:
utilizing a combined vascular and closure sheath comprising a vascular sheath having a vascular sheath lumen, a proximal end and a distal end, and an exterior surface, the vascular sheath being generally cylindrical, and comprising a closure sheath, being lesser in length and greater in diameter than the vascular sheath and having a proximal end and a distal end, disposed circumferentially around the exterior surface of the vascular sheath at the proximal end of the vascular sheath, thereby forming a closure sheath lumen between the exterior surface of the vascular sheath and an interior surface of the closure sheath, the closure sheath being configured operably to eject a fluid;
inserting the distal end of the combined vascular and closure sheath through the skin of a patient and into a vessel through a puncture in the vessel;
performing a diagnostic or therapeutic procedure within the vessel by advancing an apparatus through the vascular sheath lumen;
withdrawing the apparatus in the vascular sheath lumen;
ejecting the fluid from the closure sheath to the puncture in the vessel wall, and not within the vessel wall and vascular lumen; and
withdrawing the combined vascular and closure sheath.

13. The method of claim 12, wherein the combined vascular and closure sheath further comprises a fluid injection port disposed upon the closure sheath and through which a fluid is injected into the closure sheath lumen, the method further comprising:
injecting the fluid through the fluid injection port into the closure sheath lumen.

14. The method of claim 12, wherein the combined vascular and closure sheath further comprises a plurality of apertures disposed upon the distal end of the closure sheath and through which the fluid operably is ejected, the method further comprising:
ejecting the fluid through the plurality of apertures to the puncture in the vessel wall, and not within the vessel wall and vascular lumen.

15. The method of claim 12, wherein the combined vascular and closure sheath further comprises at least one radiopaque marker to provide for a correct alignment of the closure sheath relative to the vessel wall prior to ejection of the fluid through the closure sheath, the method further comprising:
utilizing a fluoroscope to recognize the at least one radiopaque marker; and
confirming alignment of the closure sheath relative to the vessel wall prior to ejection of the fluid through the closure sheath.

16. The method of claim 15, wherein the at least one radiopaque marker comprises a first radiopaque marker disposed at the distal end of the vascular sheath and a second radiopaque marker disposed on the vascular sheath immediately below the distal end of the closure sheath.

17. The method of claim 12, wherein the combined vascular and closure sheath further comprises at least one ultrasound marker to provide for a correct alignment of the closure sheath relative to a vessel wall prior to ejection of the fluid through the closure sheath, the method further comprising:
utilizing an ultrasound device to recognize the at least one ultrasound marker; and
confirming alignment of the closure sheath relative to the vessel wall prior to ejection of the fluid through the closure sheath.

18. The method of claim 17, wherein the at least one ultrasound marker comprises a first ultrasound marker disposed at the distal end of the vascular sheath and a second ultrasound marker disposed on the vascular sheath immediately below the distal end of the closure sheath.

19. The method of claim 12, wherein the fluid to be ejected from the closure sheath is a tissue sealant.

20. The method of claim 12, wherein the fluid to be ejected from the closure sheath is coagulation protein thrombin.
